(19) United States
(12) Patent Application Publication
(10) Pub. No.: US 2014/0356858 A1
(43) Pub. Date: Dec. 4, 2014

(54) Biological Tag Tracking of Agricultural Products, Foods, and Other Items

(71) Applicant: Advanced Biological Marketing, Inc., Van Wert, OH (US)

(72) Inventor: Gary E. HARMAN, Geneva, NY (US)

(73) Assignee: Advanced Biological Marketing, Inc., Van Wert, OH (US)

(21) Appl. No.: 14/288,631

(22) Filed: May 28, 2014

Related U.S. Application Data
(60) Provisional application No. 61/828,148, filed on May 28, 2013.

(51) Int. Cl. C12Q 1/68

(52) U.S. Cl. CPC C12Q 1/686; C12Q 1/6813
USPC 435/5; 435/6.12; 435/6.11

(57) ABSTRACT

The disclosure relates to compositions and methods for applying a biological tag to a product and detection of the biological tag on the product by another. The compositions and methods are useful for tracing the provenance of a product, such as a food product or an agricultural product.

### CROSS-REFERENCES TO RELATED APPLICATIONS

This application is entitled to priority to U.S. provisional patent application No. 61/828,148 filed May 28, 2013.

### BACKGROUND OF THE DISCLOSURE

The disclosure relates generally to the field of tracking of products, such as for determining the provenance of such products.

There is a long-felt need to be able to identify the source of commercial products and to trace their provenance. In particular, food and agricultural materials are preferably identifiable and traceable throughout the food chain.

DNA markers are already being used commercially for a wide array of tracking uses, owing to their ability to encode complex information in a manner that is difficult to forge, alter, or copy. By way of example, Applied DNA Sciences, Inc, (Stony Brook, N.Y.) offers a DNA-encoded marker product in which segments of DNA are isolated from botanical sources, fused to one another, and thereafter used as unique chemical markers that can be incorporated into a variety of materials such as inks, plastics, and metal coatings. The Applied DNA Sciences DNA marker product has been incorporated into inks used to print machine-readable tracking labels, for example, such as one- and two-dimensional bar codes. DNA sequences can be made in substantially unlimited combinations of codes since there are at least four bases that can be incorporated into DNA molecules, and substantially any conceivable combination of bases and sequence length can be used.

In many parts of the world, popular resistance to genetically-modified foodstuffs leads consumers to disfavor foods which include synthetic or human-modified genetic materials. For that reason, inclusion of synthetic DNA markers in foods or in products used in their manufacture or production can be undesirable.

A continuing need exists for markers or tags suitable to facilitate provenance tracking or detection for agricultural, food, and other products. The subject matter disclosed herein addresses that need.

### DETAILED DESCRIPTION

The disclosure relates to use of cells (and related structures) as biological “tags” suitable for tracking articles and their components. The highly variable, specifically detectable, and difficult-to-decipher chemical structures present in cells render cells suitable for use as tags.

Unlike existing technologies, which rely on detection of nucleic acids which occur naturally in a product, the biological tagging technology described herein involves adding to a product one or more types of cell that is normally not present in the product, the cell including a nucleic acid having a substantially unique sequence. It is the added cells and/or their component nucleic acid(s)that serve as detectable and difficult-to replicate tags.

The highly variable nature of cells is reflected especially in their nucleic acids, including in the DNA of a cell. The nucleic acids of cell are known to vary significantly among cells of various types, such as between cells of different species and even between strains of cells of the same species. In particular, it is known that the nucleic acids of non-related cells will often vary significantly in detectable ways, especially at portions of their genomes that do not encode highly-conserved products, such as proteins. It is routine in the art to characterize cells of a given type (e.g., cells belonging to a single species) by the occurrence in those cells, but in few or substantially no other cells, of particular nucleic acid sequences. It is similarly routine in the art to characterize cells of a given type by identifying multiple nucleic acid sequences which occur together in cells of that type, but which do occur together in few or substantially no other different cell types. That is, it is routine to identify occurrence of a cell of a particular type in a sample by identifying in the sample one or more nucleic acids that are characteristic of the particular cell type.

Cells of a given type are specifically detectable by many methods, most of which are routine in the art. By way of example, methods of detecting cells of a selected type include detecting nucleic acid sequences substantially unique to cells of the selected type, detecting antigenic determinants (i.e., epitopes) substantially unique to cells of the selected type, assessing culturability (e.g., ability to grow on certain media) and survivability (e.g., ability to be cultured after being subjected to certain agents or stimuli) of the cells, observation of morphology and behavior (e.g., motility) of the cells, and observation of chemical constituents (e.g., membrane lipids) of the cells. Many types of cells that can be used as described herein are readily culturable, and selective media that preferentially allows growth of the marker organism can be readily devised for virtually any cell type. Alternatively, a semi-selective medium can be used for culture of many cell types, so that cells of a selected type will have a characteristic appearance (e.g., a characteristic colony color or morphology) when cultured on the medium, and that characteristic appearance can be used to identify the presence of that cell (e.g., together with information on occurrence of one or more nucleotide sequences in a polynucleotide carried within the cells). Certain of these methods, especially including nucleotide sequence analysis and antigenicity are widely used in automated and rapid cell-identification methods.

Although it is possible to recover, identify, and reproduce cells that are present in a sample, such efforts are often time-consuming, difficult to perform, and expensive. The difficulty in deciphering and reproducing cells and especially a mixture of cells renders cells and their mixtures useful as difficult-to-reproduce tags. Cells of varying types can require profoundly different environments to support their survival, growth, and reproduction. A person who does not know the identity of biological cells in a sample a biological tag as described herein) must perform substantial work (e.g., either identifying the cells or trial-and-error cell culture experiments) in order to deduce conditions suitable for reproducing the cells. The substantiality of that work deters casual copying of biological tags described herein.

Even if a cell included in a biological tag described herein is recovered and identified and even if conditions amenable to reproduction of the cell can be determined, the usefulness of the cells for tagging purposes can be limited if the identity of the unique nucleotide sequences corresponding to the cells are not known. Although methods for determination and replication of nucleotide sequences isolated from cells are well known in the art, it is burdensome to determine which nucleotide sequences are substantially unique to that organism. The burden of such sequence determination and selection can further deter copying of biological tags described herein.

Except where context demands otherwise in this disclosure, “cells” means one or more cells of a unicellular or multicellular organism (from any of the three known biological domains, eukarya, bacteria, and archaea) including metabolically inactive and reproductive forms of such organisms (e.g., spores or gametes), or a virus capable of reproducing in such a cell, For convenience, these organisms are collectively referred to herein as “cells.”

Typically, nucleic acids contained within a biological cell exhibit greater stability (e.g., upon exposure to light, heat, chemical, or biological agents) than nucleic acids that are isolated from some or all of the cellular components with which they are normally associated in nature. For example, genomic DNA of microbes is substantially more stable than isolated genomic DNA of fragments of genomic DNA. Furthermore, use of organisms as biological tags has the advantage that either the organisms themselves or their nucleotide sequences (or both) can be replicated from the tag in tag-detection methods. Biological cells containing polynucleotides are easily produced (if the identity of the cells is known) and can be stabilized using a variety of encapsulating agents and processes if desired.

A wide variety of methods can be used to apply the biological tag described herein. By way of examples, tags can be applied to products by dipping the product into a composition that includes the tag, by spraying the composition onto the exterior of the product, by mixing the tag in the product (e.g., by mixing a tag into a yogurt product or by incorporating a tag into a solid packaging material containing the yogurt or in a liquid ink used to print on the surface of such a package), or ballistic application of the tag to the product (e.g., bombardment of a fruit with microparticles of an encapsulated tag).

A marker can be co-applied with the biological tag, for example to facilitate identification of its location by one seeking to find interrogate the tag. For this purpose there are a number of nontoxic natural compounds that fluoresce under ultraviolet light, for example. Dyes, radiolabels, reactive chemicals, or other detectable agents can be mixed with the biological tag and the mixture can be applied. Alternatively, the biological tag can be separately applied to the same article as an agent that identifies the location at which the biological tag was applied, such as a textual description or arrow pointing at that location.

An advantage of the biological tags described herein is that multiple biological tags can be applied to the same product (and even to the same portion of the same product) without eliminating the utility of the biological tags. Thus, multiple sequential custodians of an object can each mark a product with a discrete biological tag specific to the custodian, and one who interrogates the object for the presence of the biological tags is able to detect each of the discrete tags and conclude that each of the corresponding custodians applied its discrete tag to the object while it was in that custodian's possession. Furthermore, the custodian need not have knowledge that the object was previously possessed, or will subsequently be possessed, by a different custodian.

By way of example, a biological tagging system can be used to address food adulteration issues. Multiple food handlers in a food processing stream can each associate a biological tag unique to that handler (and/or unique to a particular lot of processed food) with the food during the period when it is in the handler's possession, to facilitate identification of all handlers who possessed the food. If the food is determined to be adulterated (e.g., through bacterial contamination), identification of all handlers of that food can permit comparison with the handlers of other similarly adulterated foods to highlight likely sources of the adulteration. Thus, for example, it may be possible to identify an individual butcher as the source of contamination of a ground meat product, facilitating identification of all meats processed by that butcher for recall and facilitating safe use of all meats similarly processed by butchers other than that individual butcher.

The location of biological tags applied to articles can be defined by conventions fix positioning of the tags. By way of example, for tags applied to bananas, a convention can be followed whereby banana producer apply their biological tag to the stems of bananas, shippers apply their biological tag to the distal ends of bananas, and importers their biological tag to a middle portion of bananas.

In the tags described herein, it can be preferable to use a combination of biological cells that have substantially different requirements for replication, no as to increase the amount and complexity of work that a person desiring to reproduce the tag would need to perform in order to do so. By way of example, a biological tag that includes both cells of an obligate anaerobic microorganism and cells of an Obligate aerobic microorganism can be difficult to reproduce unless the two types of cells are separately reproduced and thereafter recombined. Similarly, a biological tag that includes both a virus that replicates only in bacteria and mammalian cells that are difficult to reproduce in a medium containing bacteria can be difficult to reproduce.

The identity of the substantially unique nucleotide sequences used for identification purposes in the biological tags described herein is not critical. The sequences can be in coding or non-coding regions of the genome of the cell, in mitochondrial or chloroplast DNA, or in an exogenously-added nucleic acid construct such as a plasmid. However, it is desirable that the sequence be selected so that that it is unlikely to duplicate a sequence that occurs in cells of a different type, especially a cell of a type with which the product might be expected to have contact during normal handling of the product. If desired, the nucleotide sequence used for identification purposes in the biological tags described herein can be a synthetic (i.e., man-made) sequence designed to be substantially unique. However, synthetic molecules can be undesirable for use as tags in situations in which the tag is expected to be consumed by humans or other animals. Polynucleotides having detectable sequences and useful as tags can be replicable (e.g., part of genomic DNA) or non-replicable (e.g., a plasmid tacking a functional origin of replication for the cell) in the cells in which they occur in the biological tag.

If desired, the cells or nucleotide sequences selected for use in biological tags described herein can be selected according to certain ‘formatting’ rules whereby, for example, custodians of a first selected type apply identifying tags of a first distinct type and custodians of a second type apply identifying tags of a second distinct type. By way of example, in a system of biological tagging for an electronic instrument, i) manufacturers of a component of the instrument can tag a defined portion of the component using spores of one strain (selected to be unique for that manufacturer and including a first substantially unique genetic sequence) of an anaerobic solvent-producing bacterium, and ii) assemblers of the instrument can paint the interior of the assembled instrument with a paint that includes one of several strains of a killed virus (the strain used being selected to be unique to that assembler and including a second substantially unique genetic sequence) that infects only certain marine fishes, and iii) distributors of the finished instrument can attach to its exterior a label printed with an ink that includes encapsulated particles, the particles including a distributor-specific mixture of plant cells having substantially unique nucleotide sequences. Each of the component manufacturer, the assembler, and the distributor can be identified by the presence of their corresponding tags, regardless of whether the instrument is operable, broken, or even pulverized.

An important embodiment of the biological tagging technology described herein relates to tracking of foods using natural food-grade organisms that used in a manner analogous to use of DNA markers. Cells contain up to about 40 megabases of DNA and DNA sequences of cells of a selected type virtually always contain unique nucleotide sequences (i.e., sequences, and combinations of sequences, that do not occur in the nucleic acids of cells of any other type). Biological cells (including spores, gametes, and viruses) can be used as food grade “bar codes” (the combinations of nucleotide sequences present in cells of one or more selected types of cells corresponding to the combinations of stripes or spots in a one- or two-dimensional bar code of the type used in optically-scannable tagging methods for packages, for example).

One or more of the cells associated with a product as a biological tag as described herein can be encapsulated. Encapsulation of a cell can improve one or more of the cell's ability to remain culturable, the resistance of the cell to chemical or physical degradation, and the resistance of a polynucleotide within the cell to chemical or physical degradation. Each of these improvements can enhance detectability of the biological tag and extend the period of time during which the biological tag remains detectable. A wide variety of cell-encapsulation techniques are known, and substantially any such technique can be used. An encapsulation technique should be selected to be compatible with the anticipated environmental conditions to which the tagged product can be expected to be exposed. Alternatively, the compositions and methods used to encapsulate a biological tag can be selected to degrade under selected conditions (e.g., upon passing through the gut of an animal, upon being exposed to a temperature at or above a specific value, or upon exposure to highly non-polar solvents).

In addition to enhancing endurance and detectability of a biological tag, encapsulation of tag components can also contribute to information encoded by the tag. Multiple cells can be encapsulated together and those cells (or nucleic acids carried by the cells) can be detected as being co-encapsulated (e.g., by observation in the same encapsulation material or by being detectable only upon release from such encapsulation).

In an important embodiment, a biological tag capable of passing through all or part of the digestive system of an animal can be used to identify the provenance of a product ingested by the animal. By way of example, if a foodstuff is associated with a biological tag in such a way that the biological tag is ingested by a person when the person eats the foodstuff and the biological tag is encapsulated in a material that is not substantially degraded in the human digestive system, it is possible to recover the biological tag from digestive tract samples or bowel movements taken from the person. In this manner, the provenance of a food product (e.g., peanuts used as a peanut butter filling in a boxed assortment of candies) can be assessed in a human patient suspected of having been sickened by that product. Furthermore, even if the source of a human or animal food-borne illness is unknown, comparison of biological tags recoverable from different individuals exhibiting the illness can be compared to quickly identify common custodians of the same or different foods containing a common ingredient (e.g., peanuts used as a candy filling, peanuts sold for whole-nut consumption, and peanuts incorporated into a cooked sauce).

The identity of the product to which a biological tag as described herein is applied is not critical, The biological tags can be associated with agricultural products (e.g., applied to the exterior of seeds or mixed with a fertilizer or soil amendment composition), with foods (e.g., applied to a food product label, applied to a fruit rind or skin, or mixed with a food such as a yogurt or a baked good), with manufactured products (e.g., applied to a surface of the project or included as a component of at least one piece of the product), or with a tag, label, or package in which a product is associated.

When used to mark agricultural products, biological tags preferably endure in the environment(s) in which the agricultural products are stored and used, preferably for at least about the useful or anticipated lifespan of the agricultural product. Thus, a biological tag used to mark a crop seed should preferably endure under the conditions and for the period of time the crop seed is normally stored prior to planting. In one embodiment, the biological tag degrades and becomes substantially undetectable (or at least much less easily detectable) after the tagged product has been used. For example, it can be preferable if a biological tag used to mark a crop seed degrades significantly by about the time the planted seed has grown or completed its life cycle, or if a biological tag used to mark a fertilizer or soil amendment degrades significantly by about the time the fertilizer or soil amendment can be expected to be exhausted in use.

When used to mark food products, normal precautions incident to production and handling of food products should be observed. Thus, selection of cells or polynucleotides that can be expected to exhibit adverse biological consequences (e.g., triggering of allergies or other pathological effects, disagreeable taste, or promotion of spoilage) should be avoided in such situations. Considerations such as popular objection to synthetic nucleotide sequences or the presence of animal-derived materials in vegetable materials can also affect selection of cells and polynucleotides.

There are many types of detection systems that can be used to detect the biological tags described herein. The biological tags preferably include multiple signals that can be detected, including at least one target nucleotide sequence embodied in a polynucleotide carried in a biological cell. The multiple signals can be detected individually or together (i.e., serially or in parallel). Suitable signals include the presence or absence of detectable (e.g., visible or ultraviolet dye) markers, presence or absence of magnetic separation systems (e.g., magnetic or magnetically-attractable particles associated with the cell, such as by co-encapsulation of the particles and cells), specific culturability of the biological cells, and identity of specific DNA sequences contained within the cells. All of these systems can be used singly or in combination and kits provided with directions for any specific marker or combination of markers.

An advantage of using cells and nucleic acids in the biological tags described herein is that at least some cells and at least some polynucleotides are replicable. Thus, any combination of replicable and non-replicable cells and polynucleotides can be used in a biological tag described herein. Replicable cells and polynucleotides facilitate their own manufacture and permit amplification of biological tag signal (i.e., a small quantity of tag can be detected if it contains a replicable cell or polynucleotide and is subjected to appropriate replication-facilitating conditions). Non-replicable cells and polynucleotides can inhibit unauthorized duplication of the biological tag and undesirable cell growth on or in products. Similarly, selectively-replicable cells or polynucleotides (i.e., those that will replicate only if certain criteria are satisfied) can be used to assess whether an object associated with the biological tag was subjected to those criteria. By way of example, a biological tag that includes an organism that can replicate only at a temperature greater than 4 degrees Celsius can be mixed into a yogurt product. Increased concentration of the biological tag after shipping (relative to the concentration before shipping) can indicate that the yogurt was subject to a temperature greater than 4 degrees Celsius during shipping.

The biological tags and the reagents used to detect them can be provided by a common vendor or other party (e.g., an inventory control manager, a production manager, or a shipping manager). When this is so, neither a custodian who associates a biological tag with a product nor a tester who assesses the presence or absence of the biological tag in association with the product need have any substantial knowledge of the identity of the biological tag or the reagents used in its detection. Ignorance of custodians and testers as to the nature, content, and identity of the biological. tags and their components can improve the security of the tags by complicating any attempted forgery of the biological tags. Thus, even though each of nucleotide sequences, organism identity, chemistry of encapsulation, and inclusion of marker chemicals is individually discernable (i.e., if a counterfeiter knows that it must be discerned), a successful counterfeiter must decipher a nearly limitless combination of individual components, including the target nucleotide sequences carried by polynucleotides in a biological tag, the identity of the organism carrying the polynucleotides, the nature of any cell-encapsulating technology employed, and the presence or absence of other chemical markers. The biological tagging technology described herein thus provides not-practically-counterfeited methods of tracking product provenance.

### EXAMPLES

The subject matter of this disclosure is now described with reference to the following Examples. These Examples are provided for the purpose of illustration only, and the subject matter is not limited to these Examples, but rather encompasses all variations which are evident as a result of the teaching provided herein.

In the following examples, the term “bar code” is used interchangeably with the term “biological tag.”

### Example 1

### Formulation of Useful Bar Code Products Using Microorganisms (“Microbes”)

Microbes would be produced or purchased in pure form. An absolute requirement is stringent quality control to make certain of strain identity and purity. Several strains can be organized into a code for a specific product or location, with different strains used for other products or locations. If necessary, for stability, the strain mixtures can be encapsulated to provide long shelf life by techniques known to those skilled in the art.

It will probably be an advantage to use only small amounts of microbes and to be able to locate them on the food product. There are a number of fluorescent compounds that are safe to use in food, including quinine, riboflavin, turmeric, caffeine and other compounds. Small amounts of these can be mixed with the microbes, and this mixture sprayed as small dots onto the produce. The spots so made will be tasteless and invisible, but easily detected under a UV (black) light. The spots so identified can easily be removed from the produce. Since different compounds fluoresce in different colors, the fluorescent color can be an added component of any bar code.

However, other methods are possible, For example, the microbes can be encapsulated with a small amount of magnetic dust and the material recovered with a magnet.

Further, some produce has a paper label attached. The bar code organisms could be applied to and in this label.

Of course, it is possible to use the microbial bar codes as an overall spray without any fluorescent or other indicators, in this case, the microbial bar code would be provided over the entire volume of the food.

### Detection of the Microbial Bar Code

Once the product of interest are seen or removed, then, if desired, the microbial mix can be grown and the bar code thus amplified. Alternatively, DNA can be extracted, primers appropriate for the DNA codes and sub-codes used for amplification by polymerase chain reactions using techniques standard to the practitioners of this science; any DNA lab or forensic lab can easily detect the specific bar codes in the microbial label. Alternatively, hybridization to template DNA in a 96 well or other format can be used. Finally, the DNA so obtained by any of these techniques can be sequenced to find the exact code.

If we do not want to have organisms that can grow, we can use any biological material as the marker, For example, we could have powdered alfalfa (or any crop, but there is tots of alfalfa powder to feed cows and add this powder to our formulations for natural markers in addition to the microbes, This provides more diversity and choices among biological tag cells.

### Example 2

### Sources of Microbes for Bar Coding

There are a large number of suitable microbes that could be used for this invention. Among these are common bread or wine yeasts (Saccharomyces spp., which are Ascomycetous fungi), mushroom spores (e.g. Agaricus or Pleurotis spp., which are Basidomycetous fungi), the bacteria used in vinegar, yogurt cheese production (e.g., Acetobacter or Lactobacillus), fungi used in cheese making (e.g. Penicillium), strains of Aspergillus (Ascomyceteous fungi) and many others.

There are many hundreds or thousands of candidate microbes, These are all used in foods and are nontoxic and many or most are consumed by humans and other animals every day. Each of these contains thousands upon thousands of potential bar codes. A group of 20 or 30 could be mixed and matched to give thousands of combinations for potential bar coding.

### Example 3

### Identification of Microbial DNA for Bar Coding

These candidate organisms contain megabases of DNA and many are already sequenced. Others can be readily sequenced to give the DNA that wilt be the specific bar codes. Each strain has almost unlimited potential since the sequences can be of variable lengths and almost any combination of bases wilt be represented. All that is required is a modicum of bioinformatics to identify near-unique code sequences, and several different code sequences should be identified for each organism, Thus, each microbe would have several sequences, which serve as sub-bar codes (i.e., individual substantially unique nucleotide sequences detectable in combination). A combination of different microbes would thus contain the several bar codes for each strain, so if four strains were used as a specific bar code, then up to 20 sub-codes could be present.

The disclosure of every patent, patent application, and publication cited herein is hereby incorporated herein by reference in its entirety.

While this subject matter has been disclosed with reference to specific embodiments, it is apparent that other embodiments and variations can be devised by others skilled in the art without departing from the true spirit and scope of the subject matter described herein. The appended claims include all such embodiments and equivalent variations.

1. A method of assessing the provenance of a product by a tester, the method comprising
the tester assessing association of a first biological tag with the product,
wherein the first biological tag includes a first cell of a selected type, the first cell comprising a first polynucleotide having a first sequence substantially unique to cells of the first type,
presence of the first biological tag being characteristic of products that were previously in the custody of a first custodian,
whereby association of the first biological tag with the product is an indication that the provenance of the product includes custody of the product by the first custodian.

2. The method of claim 1, further comprising
the tester assessing association of a second biological tag with the product,
wherein the second biological tag includes a second cell of a selected type, the second cell comprising a second polynucleotide having a second sequence substantially unique to cells of the second type,
presence of the second biological tag being characteristic of products that were previously in the custody of a second custodian,
whereby association of the second biological tag with the product is an indication that the provenance of the product includes custody of the product by the second custodian.

3. The method of claim 1, further comprising
the tester assessing association of a second biological tag with the product,
wherein the second biological tag includes a second cell of a selected type, the second cell comprising a second polynucleotide having a second sequence substantially unique to cells of the second type,
presence of the second biological tag also being characteristic of products that were previously in the custody of the first custodian,
whereby association of both the first and the second biological tags with the product is an indication that the provenance of the product includes custody of the product by the first custodian.

4. The method of claim 3, wherein the tester assesses the presence or absence of multiple biological tags corresponding to multiple potential custodians of the product.

5. The method of claim 4, wherein the tester assesses the presence or absence of multiple biological tags corresponding to each of the potential custodians,

6. The method of claim 1, wherein the tester and the first custodian agree to a manner of associating the first biological tag with the product prior to the tester assessing association of the first biological tag with the product.

7. The method of claim 1, wherein the first cell is selected from the group consisting of a vegetative cell, a dormant cell, a killed cell, a spore, a gamete, and a virus.

8. The method of claim 1, wherein the biological tag further comprises a detectable agent selected from the group consisting of a visible pigment and a fluorescent dye.

9. The method of claim 1, wherein the first cell is associated with the product in a replicable form.

10. The method of claim 9, wherein the tester assesses association of the first biological tag with the product by subjecting at least a portion of the product to conditions under which the first cell is replicable and thereafter detecting the presence or absence of the first polynucleotide.

11. The method of claim 9, wherein the first cell becomes substantially unable to replicate if it is exposed to a selected stimulus selected from the group consisting of a temperature greater than a maximum temperature and exposure to a chemical agent

12. The method of claim 1, wherein the tester assesses association of the first biological tag with the product by subjecting at least a portion of the product to conditions under which the first polynucleotide is amplifiable and thereafter detecting the presence or absence of the first polynucleotide.

13. The method of claim 12, wherein the first sequence occurs naturally in cells of the first type.

14. The method of claim 12, wherein the first sequence is a synthetic sequence introduced into cells of the first type.

15. The method of claim 12, where the tester assesses association of the first biological tag with the product by subjecting the portion to conditions under which each of a plurality of target polynucleotides is amplifiable, each of the target polynucleotides having a target sequence substantially unique to cells of the first type and thereafter detecting the presence or absence of each of the target polynucleotides.

16. The method of claim 3, wherein the tester assesses association of each of the first and second biological tags with the product by subjecting at least a portion of the product to conditions under which each of a plurality of first target polynucleotides and a plurality of second target polynucleotides is amplifiable, each of the first target polynucleotides having a target sequence substantially unique to cells of the first type each of the second target polynucleotides having a target sequence substantially unique to cells of the second type, and thereafter detecting the presence or absence of each of the first and second target polynucleotides.

17. A method of assessing the provenance of a product by a tester, the method comprising
the tester assessing association of a plurality of biological tags with the product,
wherein each biological tag includes a cell of a selected type, that cell comprising a polynucleotide having a sequence substantially unique to cells of that type,
presence of each of the biological tags being characteristic of products that were previously in the custody of a first custodian,
whereby association of one or more of the biological tags with the product is an indication that the provenance of the product includes custody of the product by the first custodian.

18. A biological tag for tracking provenance of a product, the tag comprising
a first cell of a selected type, the first cell comprising a first polynucleotide having a first sequence substantially unique to cells of the first type,
a second cell of an independently selected type, the second cell comprising a second polynucleotide having a second sequence substantially unique to cells of the second type, and
a binder for fixedly attaching the first and second cells to the product.

19-22. (canceled)

23. A method of detectably recording provenance of a product, the method comprising
a first custodian associating with the product a first biological tag that includes a first cell of a selected type, the first cell comprising a first polynucleotide having a first sequence substantially unique to cells of the first type,
whereby a tester can assess whether the provenance of the product included custody of the product by the first custodian by assessing the presence or absence of the first biological tag in association with the product.

24-27. (canceled)

28. A method of tracking provenance of a product that is handled by multiple custodians, the method comprising
providing to each custodian a discrete biological tag for association by the custodian with the product while it is in the custodian's possession,
wherein each biological tag includes at least one cell of at least one selected type, the cells of each selected type comprising a polynucleotide having a sequence substantially unique to cells of the selected type, and
assessing the presence or absence of at least one of the biological tags provided to the custodians associated with the product to determine that the product was previously in the custody of one of the custodians.

29. (canceled)
